(19) United States
(12) Patent Application Publication
(10) Pub. No.: US 2025/0325173 A1
(43) Pub. Date: Oct. 23, 2025

(54) BENDABLE SHAFT FOR A MEDICAL HAND-HELD INSTRUMENT

(71) Applicant: Aesculap AG, Tuttlingen (DE)

(72) Inventors: Thomas Hagen, Tuttlingen (DE); Simone Hermle, Villingen-Schwenningen (DE); Ralf Pfister, Trossingen (DE); Lukas Hahn, Sigmaringen (DE); André Buerk, Villingen-Schwenningen (DE)

(21) Appl. No.: 18/292,428

(22) PCT Filed: Jul. 20, 2022
(86) PCT No.: PCT/EP2022/070333

§ 371 Date: Dec. 18, 2024

(30) Foreign Application Priority Data
Jul. 27, 2021 (DE) 10 2021 119 386.9

(51) Int. Cl. A61B 1/005; A61B 1/00

(52) U.S. Cl. CPC A61B 1/0057; A61B 1/00128

(57) ABSTRACT

A shaft of or for a medical hand instrument includes a distal shaft section having a first end face and a proximal shaft section having a second end face. The first end face and second end face face one another. At least one of the end faces is set at an angle of incidence not equal to 90° with respect to the longitudinal axis of the shaft, so that different shaft shapes result depending on the relative rotational position of the two shaft sections.

### TECHNICAL FIELD

The present disclosure relates to a shaft of or for a medical hand instrument.

In surgical technique, it is advantageous to bend the distal shaft portion of the shaft of a medical hand instrument. This allows surgeries to be performed in a small space, for example in spinal surgeries.

### PRIOR ART

Instruments that allow the distal tip to be bent have been known for some time in the field of surgical robots. This enables precise movement of the instruments in the tightest of spaces. However, in these instruments, no rotating tools are bent. One example of this is the “Da Vinci” surgical robot from Intuitive Surgical.

There are various bendable medical devices available on the market. For example, Human Xtensions has developed a bendable forceps. A surgeon can use hand-held robotic instruments to translate “rough” hand movements into delicate movements at the tip of the instrument. In this instrument, an instrument tip can be bent over a flexible area that extends over a length of approx. 20 mm. The flexible area is supported by a type of plastic stent. Adjustment is performed by way of wire strands that are guided past the outside of the stent. The disadvantage here is the length of the bending area and the very flexible tip. The flexibility is due to the plastic stent and the wire strands.

Furthermore, there are already manufacturers of bendable milling handpieces/medical hand instruments. These are primarily used in endoscopic procedures on the spine and enable minimally invasive techniques as well as easy treatment of structures that are difficult to access in this area. The joint constructions of these milling handpieces have a rather open design and a certain degree of flexibility in angulation. This means that the angle changes slightly when pressure is applied to the tip of the milling cutter. Furthermore, only angles of up to 36° are possible with these cutting handpieces. An example of this are Joimax milling handpieces.

There are bendable cordless screwdrivers with an inclined plane. In the field of DIY tools, there are cordless screwdrivers with a bendable head. These have a similar pivot joint as the disclosure described below and have very good force absorption of angulation (rigid joint). Depending on the angulation of the rotation plane, angulations of up to 90° are possible. However, the cordless screwdrivers have to be adjusted from the outside. They thus do not have internal control.

There are also high-speed milling handpieces/medical hand instruments with bent shafts. Thanks to interchangeable shafts, a handpiece can be selected from three variants: 0°, 7.5° and 15° angulation. The biggest disadvantage is the large bending radius over which bending is realized. This takes up a lot of space and limits the options for action in the surgical field. Moreover, the angle cannot be adjusted intraoperatively. Due to the fixed angulation, endoscopic operations with the bent shafts are not possible as they cannot be inserted into the straight working channel of the endoscope. Furthermore, the maximum angulation of 15° is not particularly large.

Milling cutters with bendable heads are known from the disclosures DE 10 2017 010 033 A1 and U.S. Pat. No. 10,178,998 B2. Both solutions are implemented via fork joints.

### SUMMARY OF THE DISCLOSURE

The task of the disclosure therefore consists in overcoming the disadvantages of the prior art and providing a shaft for a medical hand instrument in which a distal shaft section can be bent during operation and the angle between the distal shaft section and a proximal shaft section does not change even under load.

According to the disclosure, this task is solved by a shaft of or for a medical hand instrument having the features of claim 1. Advantageous further embodiments of the disclosure are the subject of the accompanying sub-claims.

Accordingly, the disclosure relates to the shaft of or for a medical hand instrument comprising a distal shaft section and a proximal shaft section, the respective end faces of which facing each other. At least one of the end faces is set at an angle of incidence not equal to 90° with respect to the respective longitudinal axis of the shaft, so that different shaft shapes result depending on the relative rotational position of the two shaft sections.

In other words, the shaft has a bendable distal shaft section. The distal shaft section and the proximal shaft section both have an inclined end/an angled end face with respect to the respective shaft section axis. In other words, one end/end section/end face of the distal and proximal shaft section is not straight, but beveled/angled. The beveled/angled end sections/end faces each have essentially the same angle of incidence. Therefore, the bevels match each other in such a way that the proximal and the distal shaft sections form a straight shaft/tube in a specific relative rotational position. If the distal shaft section now rotates about its longitudinal axis relative to the proximal shaft section and the proximal shaft section remains stationary, the distal shaft section is inevitably bent by the angled end faces/end sections.

The solution described above has the following advantages:
The distal shaft section of the shaft can be continuously adjusted.
The control of the rotary mechanism is fully integrated into the shaft and can be adjusted via a rotary sleeve at the proximal end section of the shaft, which is not described in detail.
The specifically designed pivot joint is very stable, smooth-running and completely insensitive to external bending forces. This enables a very precise position of the distal section of the shaft, which is not changed either by any cutting forces. This is a decisive advantage when using robot-controlled techniques.
High precision and a low risk of error are the most important arguments here.

The distal shaft section of the medical hand instrument is bendable during operation. This means that it is possible to bend the distal shaft section during surgery. With the distal shaft section resting on the inclined/angled end face of the proximal shaft section, the distal shaft section is firmly mounted.

The medical hand instrument preferably has a setting dial, the (angled) proximal shaft section, the distal shaft section that is bendable relative to it, a flexible milling cutter and a bearing. The proximal shaft section has a fixed outer tube, a ring gear (with internal teeth), an eccentric locking bush and a pinion (with external teeth that mesh with the internal teeth of the ring gear) that is in meshing engagement with the ring gear. The relatively bendable distal shaft section further preferably has a flexible transmission element, an adjusting bush with a driving pin and a distal shaft tip. The distal shaft section is preferably mounted with the bearing on the proximal section. The flexible milling cutter is preferably guided by the proximal shaft section and the distal shaft section and is bendable together with the distal shaft section. The adjusting bush is preferably joined to the pinion by the flexible transmission element in such a way that a rotational movement of the pinion is transmitted to the adjusting bush. The adjusting bush preferably transmits the rotation through the drive pin to the distal shaft tip. The rotation of the distal shaft section relative to the angled proximal shaft section causes the distal shaft section to be bent.

According to another preferred feature of the disclosure, the proximal shaft section has the ring gear with the internal teeth meshing with the external teeth of the pinion. The proximal shaft section preferably has the stationary outer tube with the angled end face. The ring gear is pivoted in the fixed outer tube about its longitudinal axis. The ring gear is preferably operable from the proximal end section of the shaft and has the internal teeth. The internal teeth mesh with the external teeth of the pinion. In other words, the internal teeth are in operative engagement with the external teeth. As a result, rotation of the ring gear is transmitted to the pinion.

According to a further preferred feature of the disclosure, the pinion is connected to the adjusting bush in the distal shaft section in a rotationally transmitting manner by a flexible transmission element. The flexible transmission element is preferably a rotary shaft or a sheet metal (strip) which rotates concentrically or also in an orbital manner (i.e. on an orbit) about a longitudinal axis. The pinion is joint to the flexible transmission element. The connection can be implemented, for example, by welding and/or gluing or another detachable or non-detachable joining technique. The adjusting bush is preferably attached to the side of the flexible transmission element facing away from the pinion. This connection, too, can also be implemented by welding or gluing. The flexible transmission element transmits the rotation of the pinion to the adjusting bush.

According to a further preferred feature of the disclosure, the adjusting bush has the drive pin, which is positively connected to the distal shaft tip and transmits the rotation of the adjusting bush to the distal shaft tip. The adjusting bush is preferably connected to the distal shaft tip by the drive pin. The rotation of the adjusting bush is thereby transmitted to the distal shaft tip and the distal shaft tip is rotated. Ultimately, the distal shaft tip thus is preferably rotated with the ring gear. Due to the rotation of the distal shaft tip, the angled end faces of the distal shaft section and the proximal shaft section are positioned against each other in such a way that the distal shaft section is bent.

According to a further preferred feature of the disclosure, the proximal shaft section has an eccentric locking bush. The eccentric locking bush is preferably mounted in the outer tube. The eccentric locking bush presses the pinion against a side of the outer tube opposite the eccentric locking bush. The pinion is preferably on the side of the shaft into which the distal shaft section is not bent. The pinion is driven by the ring gear and rotates in the eccentric locking bush. This means that the pinion is always located on the side of the outer tube. As the pinion is preferably arranged on the side into which the distal shaft section does not bend, the flexible transmission element is further away from the flexible milling cutter.

According to a further preferred feature of the disclosure, the flexible transmission element is a flexible spring plate preferably with a laterally attached ball. The flexible transmission element can be configured as a wobbling sheet metal/sheet metal circulating in an orbit. When the spring plate rotates with the pinion, it does not rotate about its own longitudinal axis, but wobbles about a longitudinal axis of the pinion. As a result, the spring plate is furthest away from the flexible milling cutter in the 45° bent position. As a result, the risk of collision is minimized, and the structure of the shaft can be executed smaller.

According to a further preferred feature of the disclosure, the ball of the flexible spring plate is received in a spherical receiving groove in the pinion and the side of the spring plate opposite the ball is preferably connected to the adjusting bush.

According to a further preferred feature of the disclosure, the ball of the flexible spring plate is received in a spherical receiving groove in the pinion and the side of the flexible spring plate opposite the ball is preferably connected to the pinion.

The ball preferably is movably accommodated in the receiving groove. The ball preferably is positively fixed in the receiving groove. However, the ball can move in a longitudinal direction of the receiving groove. The receiving groove can be fastened in the pinion or in the adjusting bush. On the side of the spring plate opposite the ball the spring plate preferably is welded or glued to the corresponding component.

According to a further preferred feature of the disclosure, the flexible transmission element is a silicone hose. Preferably, the silicone hose likewise is connected to the adjusting bush and the pinion. Preferably, the silicone hose is fastened to the adjusting bush and the pinion by gluing.

According to another preferred feature of the disclosure, the flexible transmission element is a flexible metal gaiter. The flexible metal gaiter has folds that resemble the folds of an accordion or foot pump. This makes the metal gaiter flexible and stretchable.

According to another preferred feature of the disclosure, the flexible transmission element is a flexible metal tube. The metal tube preferably has recesses or a gap geometry, which make the metal tube flexible.

Both solutions with metal tubes as the basis have the following advantages:
The metal tubes have high torsional rigidity with good bending properties at the same time. This enables very precise adjustment and/or stable positioning of the distal shaft section.
The torsional rigidity of the gap geometry is achieved through a special arrangement of the gaps.
There is no interruption of the contour in the direction of rotation and therefore no backlash.
Both solutions with metal tubes as the base can be welded or glued to the adjusting bush or pinion.

According to another preferred feature of the disclosure, a bend angle between the proximal shaft section and the distal shaft section is twice as large as the angle of incidence of the angled end faces. In the bent state, the angled end faces are in contact with each other in such a way that the angles of incidence add up. As both end faces have the same angle of incidence, the bend angle is twice as large as the angle of incidence. This doubling results in large bend angles without requiring large angles of incidence.

According to a further preferred feature of the disclosure, the bend angle between the proximal shaft section and the distal shaft section has a maximum and the angle of incidence becomes smaller again when the distal shaft section is rotated further. When the distal shaft section is rotated further, the angled end faces are no longer directly perpendicular to one another. Therefore, the bend angle decreases again with further rotation.

According to a further preferred feature of the disclosure, the adjusting bush is made of a sliding bearing material, preferably PTFE or POM, and/or has a coating with PTFE. The adjusting bush preferably rotates in a receiving bore of the outer tube. Preferably, no other bearing is arranged in the receiving bore. The adjusting bush must therefore slide. Due to the manufacture from the sliding bearing material, the adjusting bush has a lower friction with respect to the receiving bore.

According to a further preferred feature of the disclosure, the distal shaft tip is made of a plastic with good sliding properties, preferably PTFE or POM. If no bearing is fastened between the outer tube and the distal shaft tip, the distal shaft tip must be rotatable with respect to the outer tube. This is ensured by the material selection of the distal shaft tip.

According to a further preferred feature of the disclosure, the distal shaft tip is made of a flexible plastic. The distal shaft tip is manufactured such that it is bendable. This allows the distal shaft tip to form an undercut with the outer tube, which fixes the distal shaft tip to the proximal shaft section. For assembly, the distal shaft tip is bent open and locked in place with the outer tube.

According to a further preferred feature of the disclosure, a desired angular position is set manually or by motor via a setting dial. The setting dial is preferably located at the proximal end of the hand instrument. This allows a user to set a desired angle. For example, the user can turn a small wheel, which represents the adjustment element, or the user can set the desired angle using a lever, joystick or the like.

According to a further preferred feature of the disclosure, the bearing is a solid ball bearing. A solid ball bearing reduces the friction in the bearing. As a result, there are fewer losses when adjusting the angle. Likewise, the necessary play for smooth adjustability can be minimized, and thus the precision of the distal tip can be increased.

According to a further preferred feature of the disclosure, the roller bearing is a ball bearing with at least, preferably exactly, three balls. The use of three or more balls increases the friction in the roller bearing. However, it is advantageous that the roller bearing can be assembled more quickly since fewer balls have to be filled in, and that the roller bearing is cheaper.

### SHORT DESCRIPTION OF THE FIGURES

FIG. 1 shows a shaft according to a first embodiment in a straight shaft form;

FIG. 2 shows the shaft according to the first embodiment in which a distal shaft section is angled by 22.5° compared to a proximal shaft section;

FIG. 3 shows the shaft according to the first embodiment in which the distal shaft section is angled at 45° compared to the proximal shaft section;

FIG. 4 shows a longitudinal section through the straight shaft;

FIG. 5 shows a longitudinal section through the shaft angled by 22.5°;

FIG. 6 shows a longitudinal section through the shaft angled by 45°;

FIG. 7 shows a cross-section through the proximal shaft section;

FIG. 8 shows a cross-section through a roller bearing arranged between the proximal shaft section and the distal shaft section;

FIG. 9 shows an isometric view of a spring plate with a ball;

FIG. 10 shows a side view of the spring plate;

FIG. 11 shows the spring plate in the straight shaft;

FIG. 12 shows the spring plate with the shaft angled by 45°;

FIG. 13 shows a top view of the spring plate in the shaft angled by 22.5°;

FIG. 14 shows the twisted spring plate in the shaft angled by 22.5°;

FIG. 15 shows a cross-section through the proximal shaft section;

FIG. 16 shows an isometric view of a flexible silicone hose according to the first embodiment;

FIG. 17 shows a side view of the flexible silicone hose according to the first embodiment;

FIG. 18 shows an isometric view of a flexible metal gaiter according to a third embodiment;

FIG. 19 shows a side view of the flexible metal gaiter according to the third embodiment;

FIG. 20 shows an isometric view of a flexible metal tube according to a fourth embodiment;

FIG. 21 shows a side view of the flexible metal tube according to the fourth embodiment;

FIG. 22 shows a longitudinal section through the straight shaft with a roller bearing according to a fifth embodiment;

FIG. 23 shows a cross-section through the roller bearing according to the fifth embodiment;

FIG. 24 shows a longitudinal section through a straight shaft according to a sixth embodiment;

FIG. 25 shows a cross-section through a distal shaft section according to the sixth embodiment;

FIG. 26 shows a shaft according to a seventh embodiment; and

FIG. 27 shows a hand instrument with the bendable shaft according to the disclosure.

### DESCRIPTION OF THE EMBODIMENTS

Subsequently, preferred embodiments of the present disclosure are described based on the accompanying figures.

### First Embodiment

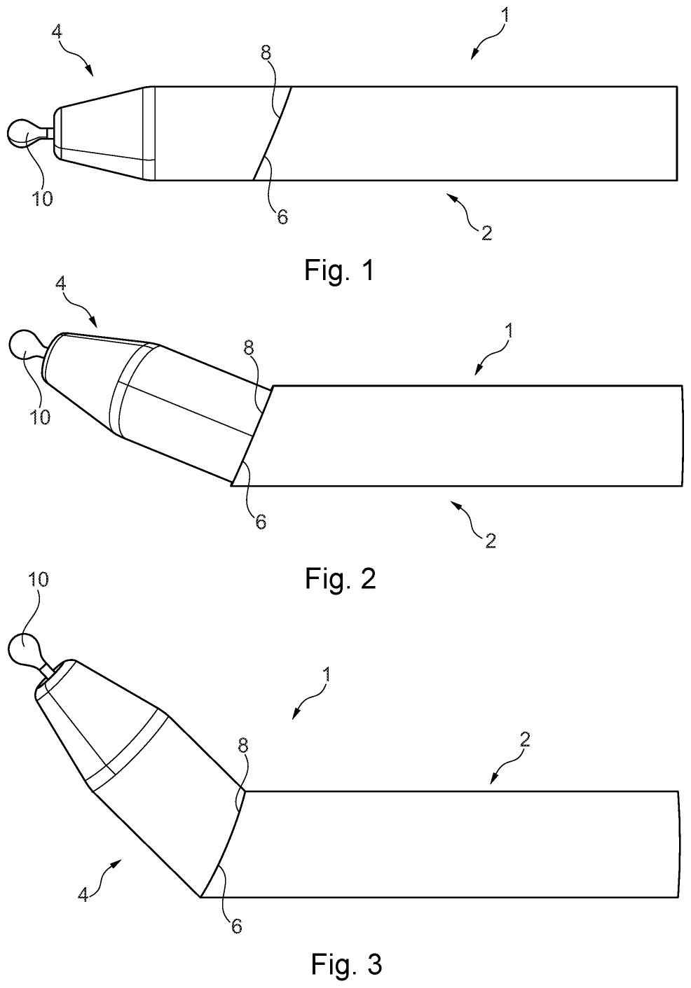

FIG. 1 shows a shaft 1 of or for a medical hand instrument in a straight shaft form. That is, a proximal shaft section 2 of the shaft 1 and a distal shaft section 4 of the shaft 1 are arranged in a line or have an angle of 0° with respect to each other. The proximal shaft section 2 is essentially tubular and has an end face 6 at its distal end section that is angled in the longitudinal axis of the shaft. The distal shaft section 4 likewise is approximately tubular. The tube converges towards the distal end to form a point. At its proximal end section, the distal shaft section 4 has an end face 8 angled in the longitudinal axis of the shaft 1. A flexible milling cutter 10 protrudes from the distal end of the shaft 1. The flexible milling cutter 10 runs along the longitudinal axis of the shaft. It is obvious that a drill or other medical tool can protrude from the shaft 1 instead of the flexible milling cutter 10.

The angled end faces 6, 8 each have an angle of incidence of preferably 22.5° to a plane normal to the longitudinal axis of the shaft. When the shaft 1 is in a straight shaft form or extended, the two angled end faces 6, 8 are displaced to each other in such a way that the long ends of the angled end faces 6, 8 are opposite each other in relation to the longitudinal axis. The angled end faces rest on each other. The angled end faces do not necessarily have to have an angle of incidence of 22.5°. Angles of attack of, for example, 10°, 18°, 30°, 45° or any other angle of incidence are also possible.

FIG. 2 shows the shaft 1, whereby the distal shaft section 4 is angled by 22.5° with respect to the proximal shaft section 2. Compared to the position in FIG. 1, the distal shaft section 4 is rotated through 90° about its own longitudinal axis relative to the proximal shaft section 2. The angled end faces 6, 8 do not lie completely/flat on top of each other. The angled end face 8 is rotated with the distal shaft section 4 through 90° relative to the angled end face 6 of the proximal shaft section 2, so that a long end of the angled end face 8 protrudes beyond the angled end face 6. Due to the angle of incidence of the angled end face 6, the distal shaft section 4 protrudes upwards relative to the proximal shaft section 2. The flexible milling cutter 10 is bent together with the distal shaft section 4.

FIG. 3 shows the shaft 1, whereby the distal shaft section 4, with a respective angle of incidence of the two end faces by 22.5°, consequently is angled with respect to the proximal shaft section 2 by 45°. Compared to the position in FIG. 1, the distal shaft section 4 is rotated through 180° about its own longitudinal axis. In this position, the angled end faces 6, 8 again lie completely/flat on top of each other. However, due to the rotation of the distal shaft section 4, the long ends of the angled end faces 6, 8 are located adjacent to each other. The angles of incidence of the angled end faces 6, 8 thus add up. As a result, the distal shaft section 4 is angled by twice the angle of incidence of the angled end faces 6, 8 compared to the proximal shaft section 2.

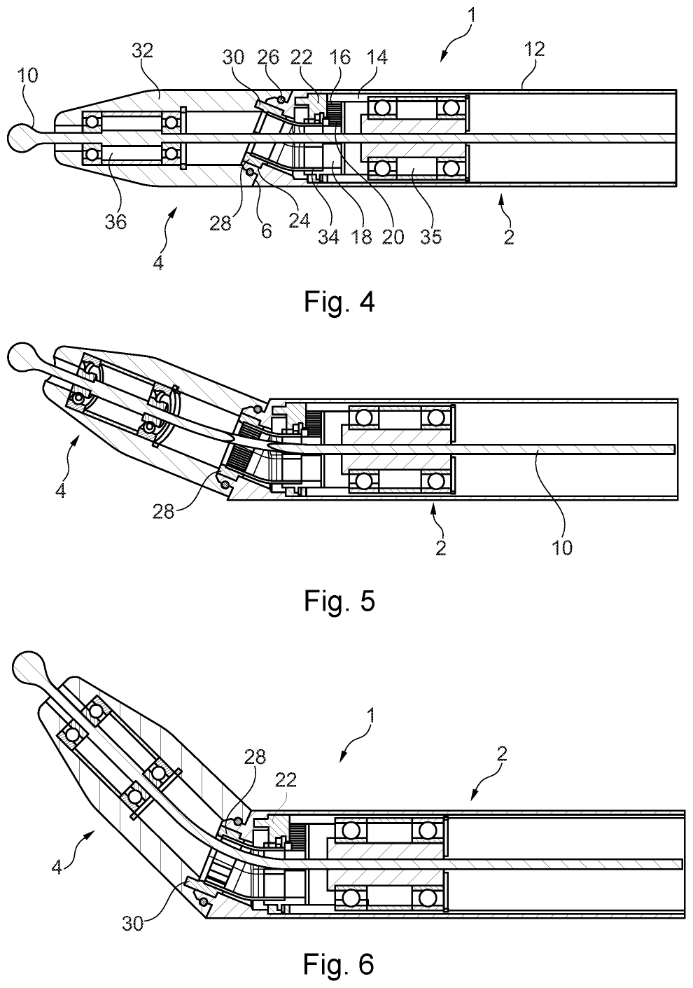

FIG. 4 shows a cross-section of the extended (straight) shaft 1 through a longitudinal axis. The proximal shaft section 2 has a stationary outer tube 12, a ring gear 14 with internal teeth 16, a pinion 18 with external teeth 20 and an eccentric locking bush 22. The ring gear 14 is located within the outer tube 12 and the longitudinal axis of the outer tube 12 corresponds to the longitudinal axis of the ring gear 14. The outer tube 12 and the ring gear 14 thus are arranged concentrically. The ring gear 14 is connected to a proximal setting dial (not shown) and rotates with the setting dial. A user can set a desired angular position on the setting dial. The angular position can be set either manually, or assisted by a motor. The proximal setting dial can also have a locking device (not shown) (for example a ball pressure piece, a clamping screw or a locking ring), which blocks the adjustment of the distal shaft section 4. This ensures that adjustment only takes place through active, intentional action. The outer tube 12 does not move. The distal end of the outer tube 12 has the angled end face 6. The distal end of the outer tube 12 moreover has a receiving bore 24 and a receiving pin for a roller bearing 26. The outer tube has a groove for the balls of the roller bearing 26. The distal shaft section 4 is mounted on the roller bearing 26.

The internal teeth 16 mesh with the external teeth 20 of the pinion 18. As a result, a rotation of the ring gear 14, which is controlled by the setting dial, is transmitted to the pinion 18. The direction of rotation of the pinion 18 is opposite to the direction of rotation of the ring gear 14. The pinion 18 is driven by the ring gear 14, but the pinion rotates in the eccentric locking bush 22. The locking bush 22 is disposed eccentrically to the ring gear 14. In other words, although the longitudinal axis of the eccentric locking bush 22 is parallel to the longitudinal axis of the ring gear 14, the longitudinal axes do not lie on top of each other.

The distal shaft section 4 has an adjusting bush 28 with a driving pin 30 and a distal shaft tip 32. The distal shaft tip 32 is mounted on the roller bearing 26. The adjusting bush 28 is mounted in the receiving bore 24 of the proximal shaft section 2. The driving pin 30 of the adjusting bush 28 engages positively in the distal shaft tip 32. The adjusting bush 28 is connected to the pinion 18 via a flexible silicone hose 34 in such a way that a rotation of the pinion 18 is transmitted to the adjusting bush 28. The flexible silicone hose 34 is a flexible transmission element in accordance with the claim. The flexible silicone hose 34 is fastened to the adjusting bush 28 and the pinion 18, for example by welding or gluing. Since the adjusting bush 28 is positively connected to the distal shaft tip 32 via the driving pin 30, a rotation of the adjusting bush 28 is transmitted to the distal shaft tip 32.

The flexible milling cutter 10 extends both through the proximal shaft section 2 and through the distal shaft section 4. The flexible milling cutter 10 is mounted in the proximal shaft section 2 and in the distal shaft section 4 through roller bearings 35, 36. The flexible milling cutter 10 can be bent with the distal shaft section 4.

This arrangement allows the distal shaft section 4 to be continuously adjusted between 0 and 45 degrees. The full roller bearing 26 ensures that smooth and jerk-free adjustment (no stick/slip effect) is possible even when the instrument tip is under load.

For mounting the shaft 1, the locking bush 16 is first inserted into the outer tube 12. Subsequently, the ring gear 14 with the pinion 18 is inserted into the outer tube. The bearing 34 for the flexible milling cutter 10 is then mounted and secured with a lock ring. The adjusting bush 28 with the flexible transmission element 34 is inserted into the receiving bore 24. The roller bearing 26 is placed on the outer tube 12. The distal shaft tip 32 is placed on the roller bearing 26. The driving pin 30 of the adjusting bush 28 engages positively in the distal shaft tip 32.

FIG. 5 shows a cross-section of the shaft 1 angled by 22.5°, with the distal shaft section 4 angled by 22.5° compared to the proximal shaft section 2. It should be noted that the driving pin 30 is not to be seen in this cross-sectional view, as the driving pin 30 rotates with the adjusting bush 28 and is concealed by the adjusting bush 28 in this view. The flexible milling cutter 10 is twisted with the proximal shaft section 2. The distal shaft section 4 moves on a circular path to the 22.5 degree position. The ring gear 14 and the pinion 18 each rotate in the opposite direction.

Low friction of the receiving bore 24 bearing the adjusting bush 28 is advantageous. This can be achieved by using a sliding bearing material (e.g. PTFE, POM) or a coating (e.g. PTFE) on the adjusting bush 28.

FIG. 6 shows a cross-section of bent shaft 1 with an angle of 45°. In this position, the driving pin 30 is located opposite the locking bush 22. This means that the adjusting bush 28 with the driving pin 30 has rotated through 180° from an extended position to a maximum angled position.

The 45° position represents the reverse point for this structure. The adjusting bush 28 has rotated through 180° in this position. When the ring gear 14 is rotated further, the distal shaft section 4 rotates back to the starting position (0° position). Depending on the application, this may be advantageous or also unnecessary. In the second case, the result would be a reversal of the direction of rotation to return to the starting position. As the instrument can be rotated 360 degrees around its own axis in the working channel of the endoscope, any position nevertheless can be reached.

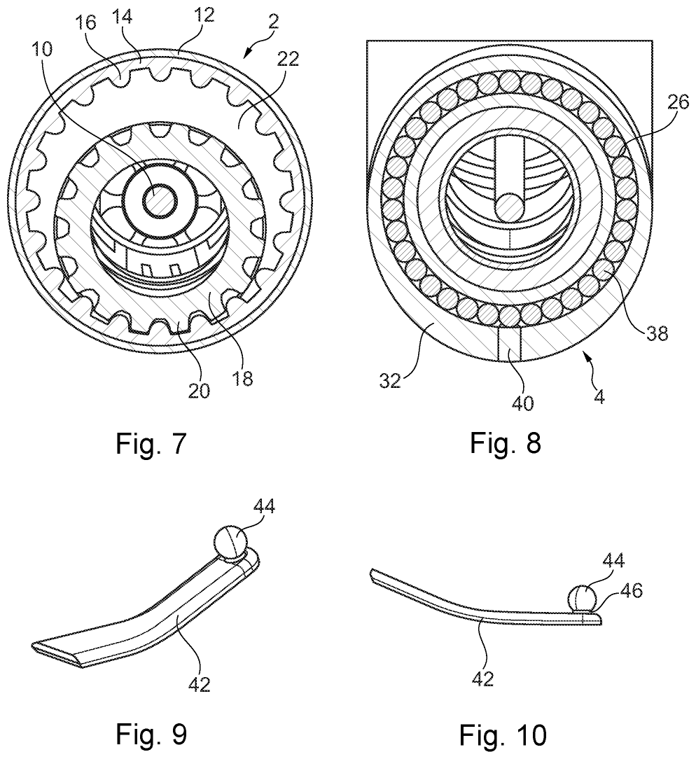

FIG. 7 shows a cross-section through the proximal shaft section 2. FIG. 7 shows the ring gear 14 with the internal teeth 16 in the outer tube 12. The pinion 18 with the external teeth 20 meshes with the internal teeth 16. The eccentric locking bush 22 is fixed to the upper side of the outer tube 12 and is in contact with the pinion 18. As a result, the pinion 18 remains on the underside of the outer tube 12 even when the ring gear 14 rotates. The underside of the outer tube 12 is the side opposite the eccentric locking bush 22. This means that the eccentric locking bush 22 presses the pinion 18 towards the underside of the outer tube 12.

FIG. 8 shows a cross-section through the distal shaft section 4 and the roller bearing 26. The roller bearing 26 is preferably a solid ball bearing. The design of the roller bearing 26 as a solid ball bearing ensures minimal friction in the roller bearing 26. For mounting the roller bearing 26, balls 38 are inserted into the groove track through the filling opening 40. A calculated clearance of 1 to 2 balls is advantageous for smooth rolling of the rolling elements. The filling opening 40 can be closed with a dowel pin and the dowel pin can be non-detachably welded.

### Second Embodiment

In a second embodiment, the flexible transmission element is implemented by way of a spring plate 42 with a laterally mounted ball 44. FIG. 9 shows the spring plate 42 in an isometric view. The spring plate 42 is flexible and has a lateral pin 46 on which the ball 44 is mounted. The pin 46 is either firmly welded to the spring plate 42, or the pin 46 is connected to the spring plate 42 so that it can rotate about its own longitudinal axis.

In the preferred embodiment, a flexible transmission element is shown. This can be implemented in different forms. The preferred variant is the spring plate 42 with the laterally mounted ball 44, which is particularly stable in position and has a high repeat accuracy. Furthermore, the spring plate 42 requires little space due to its lateral attachment and therefore offers more room for the flexible milling cutter 10 to pass through. The flexible transmission element is the flexible spring plate 42 including the laterally attached pin 46 with the ball tip. FIG. 10 shows a side view of the spring plate 42. The spring plate 42 is flexible. The spring plate 42 is bendable both about an axis that runs perpendicular to the longitudinal axis of the spring plate 42 and about the longitudinal axis of the spring plate 42.

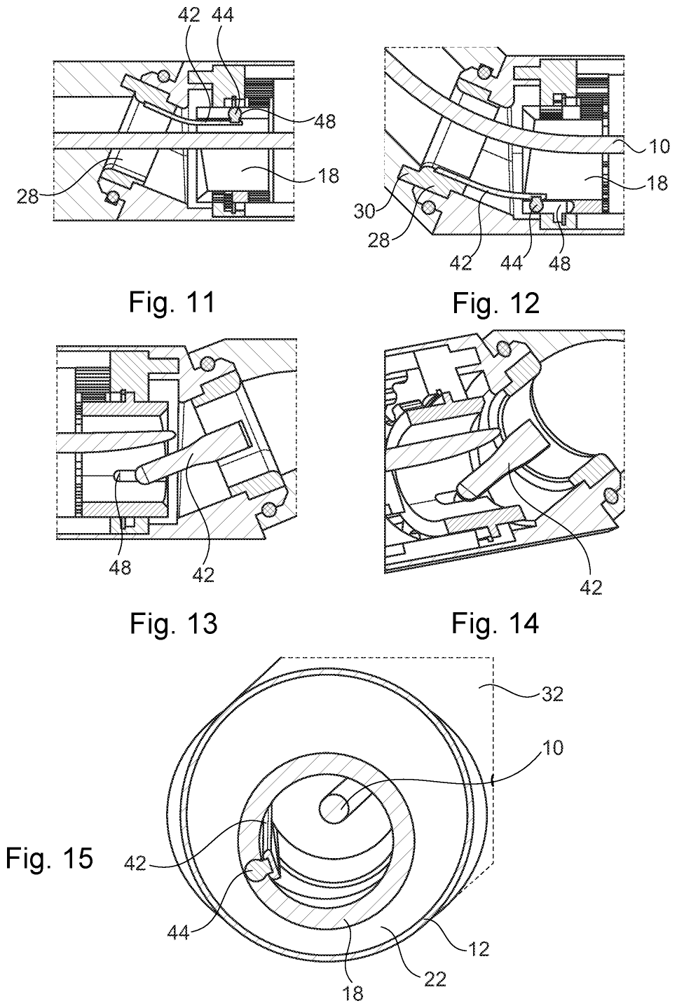

FIG. 11 shows the spring plate 42 in the straight shaft 1. It is illustrated that the ball 44 of the spring plate 42 is inserted in an elongated receiving groove 48 in the pinion 18. The receiving groove 48 runs in the longitudinal direction of the shaft 1 and the ball 44 is positively connected to the receiving groove 48. The ball 44 therefore moves with the rotation of the pinion 18 and also moves in the longitudinal direction of the receiving groove 48. The side of the spring plate 42 opposite the ball 44 is fixed to the adjusting bush 28. A rotation of the pinion 18 thus is transmitted from the spring plate 42 to the adjusting bush 28. The adjusting bush 28 rotates with the spring plate 42. The spring plate 42 is connected to the adjusting bush 28 by welding or gluing, for example. It should be noted that the receiving groove 48 can also be prepared in the adjusting bush 28. In this case, the ball 44 is connected to the adjusting bush 28 and the spring plate 42 is welded or glued to the pinion 18.

The ball 44 moves in the spherical receiving groove 48 in the pinion 18 in the longitudinal direction of the receiving groove 48. A distal fixation in the adjusting bush 28 is implemented by means of gluing or welding. The proximal fixation is omitted. Due to the positive fit of the ball 44 in the receiving groove 48, the rotary movement is transmitted to the adjusting bush 28 via the spring plate 42.

FIG. 12 shows the spring plate 42 in the shaft 1 angled by 45°, with the pinion 18 rotated through 180° around its longitudinal axis compared to the straight shaft 1. Naturally, the receiving groove 48 including the ball 44 is likewise rotated through 180°. The adjusting bush 28 also is rotated through the spring plate 42. The driving pin 30 carries the distal shaft tip 32, which also rotates through 180°. This clearly shows that the spring plate 42 is at a greater distance to the flexible milling cutter 10 in the angled state than in the extended state.

FIGS. 11 and 12 show the position of the ball 44 and the bending of the spring plate 42 in the two end positions. In the 45° position, the spring plate 42 is at the bottom. Together with an eccentric bore in the adjusting bush 28 and an eccentric chamfer on the pinion 18, the clearance for the flexible milling cutter 10 is greatest here. This is the main difference to the variants listed below and can be decisive when miniaturizing the structure.

FIG. 13 shows a top view of the spring plate 42, with the shaft 1 angled by 22.5°. The rotation of the pinion 18 causes the spring plate 42 to rotate and the adjusting bush 28 to rotate with it. This causes the spring plate 42 to be twisted. It is shown that the ball 44 moves in the longitudinal direction of the receiving groove 48 when the pinion 18 rotates in the receiving groove 48. FIG. 14 shows the spring plate 42, clearly illustrating that the spring plate 42 is twisted about its own longitudinal axis during rotation or transmission of the rotary movement from the pinion 18 to the adjusting bush 28. In the 22.5° position, it becomes apparent that the spring plate 42 rotates relative to the receiving groove 48 and also is twisted about its longitudinal axis. A corresponding flexibility of the spring plate 42 is a basic prerequisite for the function.

FIG. 15 shows a cross-section through the proximal shaft section 2 with the eccentric locking bush 22 and the pinion 18. The ball 44 is positively engaged in the receiving groove 48 and follows the rotation of the receiving groove 48. The cross-section shows that the special receiving groove 48 with its spherical shape ensures that the ball 44 of the spring plate 42 is gripped by a positive fit and must therefore follow the rotational movement of the pinion 18.

FIG. 16 shows the silicone hose 34 according to the first embodiment. The silicone hose 34 is one way of realizing the flexible transmission element. One end of the silicone hose 34 is respectively glued to the pinion 18 and the adjusting bush 28. FIG. 17 shows a side view of the silicone hose 34.

### Third Embodiment

FIG. 18 shows a flexible transmission element according to a third embodiment. The transmission element is a flexible metal gaiter 50. The metal gaiter 50 is essentially a hollow elongated metal tube. In the center, the metal tube has folds that resemble the folds of a bellows or an accordion. The folds make the metal gaiter 50 bendable or flexible. The metal gaiter 50 can be attached to the pinion 18 and the adjusting bush 28 by gluing or welding. FIG. 19 shows a side view of the metal gaiter 50.

### Fourth Embodiment

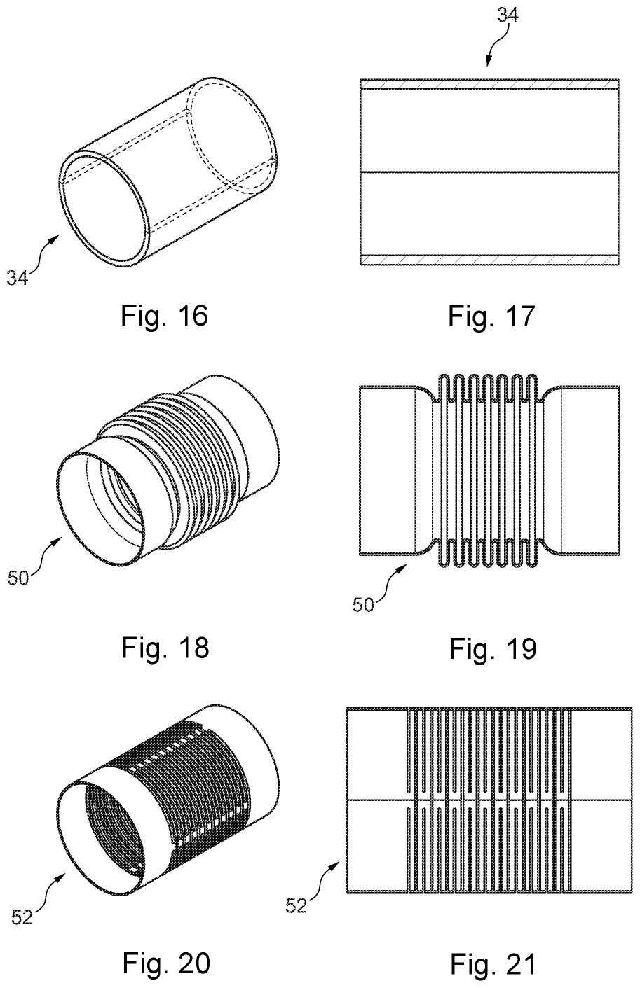

FIG. 20 shows a flexible transmission element according to a fourth embodiment. The transmission element is a flexible metal tube 52. The metal tube 52 is essentially a thin elongate metal tube. The metal tube 52 has slots/recesses that extend in the radial direction of the metal tube 52. In other words, the metal tube 52 has a slit geometry. The recesses make the metal tube 52 flexible. The metal tube 52 can be fastened to the pinion 18 and the adjusting bush 28 by gluing or welding. FIG. 21 shows a side view of the metal tube 52.

### Fifth Embodiment

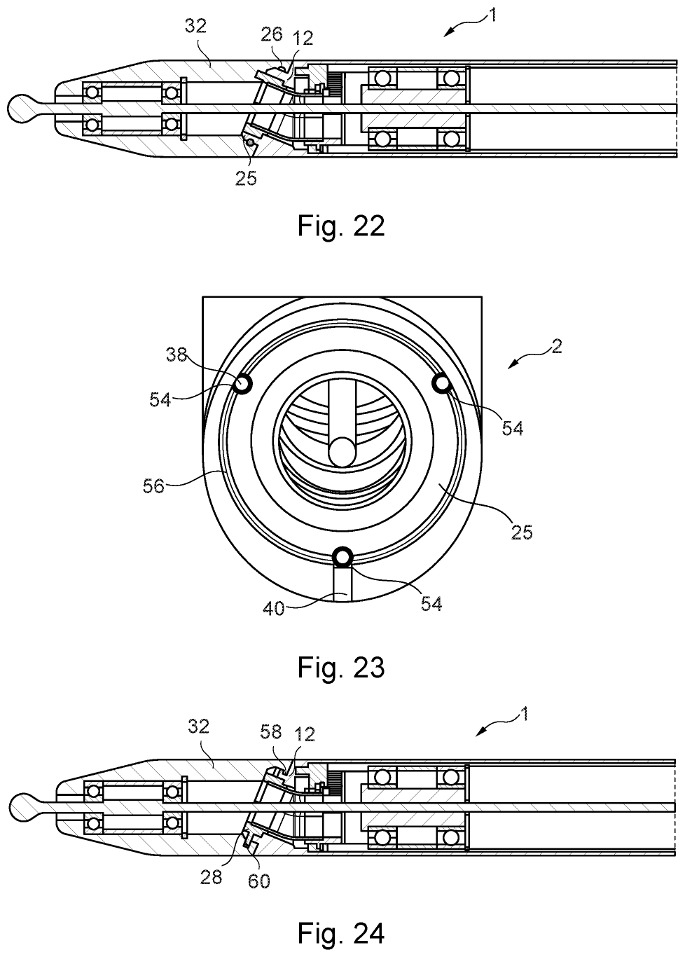

FIG. 22 shows an extended shaft 1 according to a fifth embodiment. According to the fifth embodiment, the roller bearing 26 is not a solid ball bearing, but has only three or also more balls 38. The receiving pin 25 of the outer tube 12 has three ball bores 54. The distal shaft tip 32 has a circumferential groove 56. During assembly, the balls 38 each are inserted through the filling opening 40 into the corresponding ball bore 54. The closure is performed analogously to the first embodiment. The balls 38 rotate in their respective ball bore 54. This leads to a slightly higher friction, since the rolling movement is only possible on the groove 56 of the distal shaft tip 32. However, considerably fewer balls 38 are required and assembly is faster. FIG. 23 shows a cross-section through the distal shaft section 4 and the roller bearing 26. The ball bores 54 are equally spaced in the circumferential direction.

### Sixth Embodiment

FIG. 24 shows an extended shaft 1 according to a sixth embodiment. In the sixth embodiment, there is no roller bearing between the outer tube 12 and the distal shaft tip 32. Rather, a sliding pairing is formed between the outer tube 12 and the distal shaft tip 32. The distal shaft tip 32 is fixed to the outer tube 12 by a rear catch 58. In order to be able to realize the rear catch 58, the distal shaft tip 32 must be made of a flexible material, such as plastic, for example. The distal shaft tip 32 is bent up for assembly and snapped into the projections of the outer tube 12. Rotation is transmitted by a lateral driving pin 60, as, due to the lack of balls, space has been created. The driving pin 60 protrudes from the adjusting bush 28 and rotates with the adjusting bush 28. The outer tube 12 has a recess 61 in which the driving pin 60 can be moved. The driving pin 60 extends through the recess 61 and is connected to a bulge of the distal shaft tip 32 in a form-fit manner.

If a plastic with good sliding properties (e.g. PTFE, POM) is used, smooth adjustment can be implemented even without roller bearings. This variant is particularly suitable for low-cost single-use instruments.

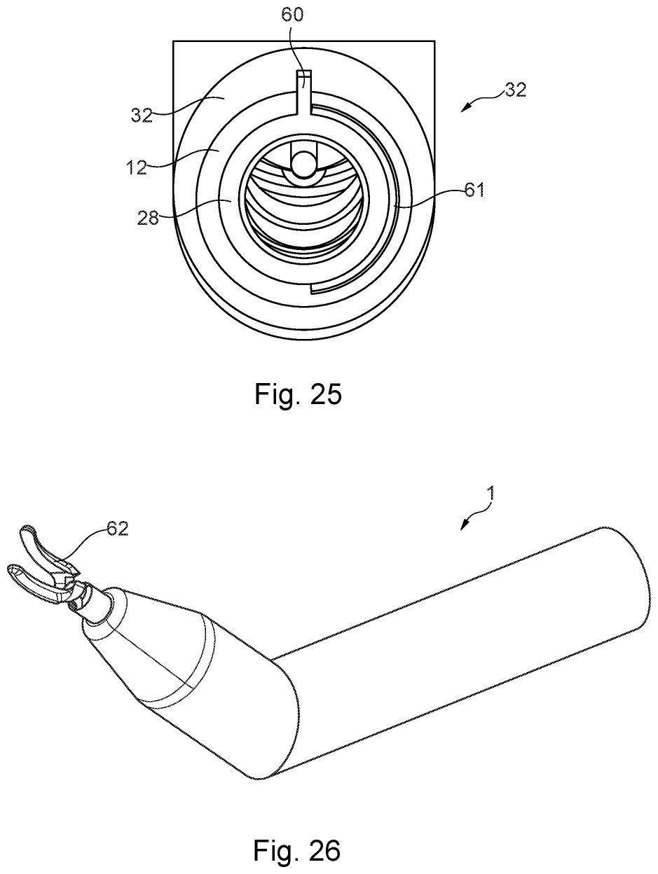

FIG. 25 shows a cross-section of the distal shaft tip 32. The adjusting bush 28 has the protruding driving pin 60, which engages in the distal shaft tip 32. The driving pin 60 pierces the outer tube 12 through the recess 61. The recess 61 covers half the circumference of the outer tube 12. The driving pin 60 transmits the rotation of the adjusting bush 28 to the distal shaft tip 32.

### Seventh Embodiment

FIG. 26 shows a bent shaft 1 according to a seventh embodiment. Here, the shaft 1 does not have a cutter, but has jaw parts or scissor blades 62 that protrude from the distal shaft section. A user can grip or fixate objects with the jaw parts 62.

In endoscopic instruments with an angle-adjustable tip, friction of the flexible control plays a subordinate role, as high frequencies for the movement of the jaws or scissor blades 62 generally are not achieved (<100 actuations per minute).

In this case, larger angulations can be achieved without unacceptable heating of the shaft. By an angled cut of 45 degrees (instead of 22.5 degrees) a maximum angulation of 90 degrees can be achieved (not shown). However, the more elliptical shape of the cut surface increases the lateral protrusion of the shaft edges in the area of the pivot joint (especially in the intermediate position halfway through the adjustment range). The protrusion can be reduced through specific curves.

Theoretically, retrograde instruments (angulation >90 degrees) would also be conceivable, but this design is not useful due to a flat cutting angle and a very large projection.

The special concept of the angular head enables particularly rigid instruments that can maintain their position even under high loads.

The flexible milling cutter 10 can have a special section for the bending area, which, on the one hand, can withstand the movement of the instrument tip and, on the other hand, can transmit the torque. This can be, for example, a thin wire, braided strands or a universal joint or similar.

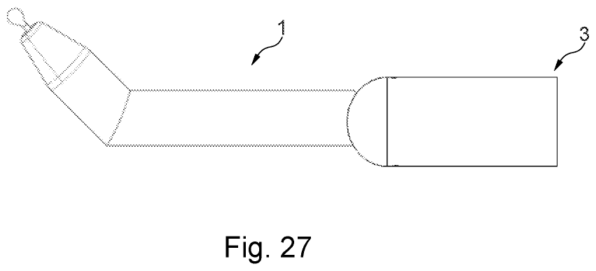

FIG. 27 shows the shaft 1 with a medical hand instrument 3. The shafts of all embodiments are suitable for the medical hand instrument 3 and can be joint to the medical hand instrument 3.

### LIST OF REFERENCE SIGNS

1 Shaft
2 Proximal shaft section
3 Medical hand instrument
4 Distal shaft section
6, 8 Angled end face
10 Flexible milling cutter
12 Outer tube
14 Ring gear
16 Internal teeth
18 Pinion
20 External teeth
22 Eccentric locking bush
26 Roller bearing
28 Adjusting bush
30, 60 Driving pin
32 Distal shaft tip
34 Flexible transmission element
38 Balls
42 Spring plate
48 Receiving groove

1. Shaft (1) of or for a medical hand instrument (3) comprising a distal shaft section (4) and a proximal shaft section (2), the respective end faces (6, 8) of which facing one another and of which at least one is set at an angle of incidence not equal to 90° with respect to the respective longitudinal axis of the shaft, so that different shaft shapes result depending on the relative rotational position of the two shaft sections (2, 4).

2. The shaft (1) according to claim 1, characterized in that the angles of incidence are equal and a straight or a bent shaft shape results depending on the relative rotational position of the two shaft sections (2, 4).

3. The shaft (1) according to claim 1 or 2, characterized in that the proximal shaft (2) has a ring gear (14) with internal teeth (16) which meshes with external teeth (20) of a pinion (18).

4. The shaft (1) according to any of claims 1 to 3, characterized in that the pinion (18) is connected to an adjusting bush (28) in the distal shaft section (4) by a flexible transmission element (34; 42; 50; 52) in a rotationally transmitting manner.

5. The shaft (1) according to any of claims 1 to 4, characterized in that the adjusting bush (28) has a driving pin (30; 60) which is positively connected to a distal shaft tip (32) and transmits the rotation of the adjusting bush (28) to the distal shaft tip (32).

6. The shaft (1) according to any of claims 1 to 5, characterized in that the proximal shaft section (2) has an eccentric locking bush (22).

7. The shaft (1) according to any of claims 1 to 6, characterized in that the flexible transmission element is a flexible spring plate (42) with a laterally attached ball (44).

8. The shaft (1) according to claim 7, characterized in that the ball (44) of the flexible spring plate (42) is received in a spherical receiving groove (48) in the pinion (18) and the side of the spring plate (42) opposite the ball (44) is connected to the adjusting bush (28).

9. The shaft (1) according to claim 7, characterized in that the ball (44) of the flexible spring plate (42) is received in a spherical receiving groove (48) in the adjusting bush (28) and the side of the flexible spring plate (42) opposite the ball (44) is connected to the pinion (18).

10. The shaft (1) according to any of claims 1 to 6, characterized in that the flexible transmission element is a silicone hose (34).

11. The shaft (1) according to any of claims 1 to 6, characterized in that the flexible transmission element is a flexible metal gaiter (50).

12. The shaft (1) according to any of claims 1 to 6, characterized in that the flexible transmission element is a flexible metal tube (52).

13. The shaft (1) according to any of claims 1 to 12, characterized in that a bending angle between the proximal shaft section (2) and the distal shaft section (4) is twice as large as the angle of incidence of the angled end faces (6, 8).

14. The shaft (1) according to any of claims 1 to 13, characterized in that the bending angle between the proximal shaft section (2) and the distal shaft section (4) has a maximum and the angle of incidence becomes smaller again with a further rotation of the distal shaft section (4).

15. The shaft (1) according to any of claims 1 to 14, characterized in that the adjusting bush (28) is made of a sliding bearing material, preferably PTFE or POM, and/or has a coating with PTFE.
